(19) United States
(12) Patent Application Publication
(10) Pub. No.: US 2025/0104807 A1
(43) Pub. Date: Mar. 27, 2025

(54) Systems And Methods For Use In Resolving Genotypic Indicators

(71) Applicant: MONSANTO TECHNOLOGY LLC, Saint Louis, MO (US)

(72) Inventors: Guilherme GOMES, Creve Coeur, MO (US); Christopher SCHLOSBERG, Chicago, IL (US)

(21) Appl. No.: 18/890,093

(22) Filed: Sep. 19, 2024

Related U.S. Application Data
(60) Provisional application No. 63/540,038, filed on Sep. 22, 2023.

(51) Int. Cl. G16B 20/20; C12Q 1/6837; G01N 21/64; G16B 40/30

(52) U.S. Cl. CPC G16B 20/20; C12Q 1/6837; G01N 21/6428; G16B 40/30; G01N 2021/6441

(57) ABSTRACT

Example systems and methods for resolving indicators associated with specific genotypes of samples are disclosed. One example computer-implemented method includes exposing a sample of an organism to multiple probes, where the multiple probes include a first type of probe configured to bind to a first target region containing a target marker with a first target allele and a second type of probe configured to bind to a second target region containing the target marker with a second target allele, the first allele different than the second allele, and detecting, by a detector, intensities of fluorescence in the sample from the multiple probes. The method also includes identifying, by a computer device, based on the detected intensities of fluorescence in the sample and using a generative model, a genotype at the target marker of the organism and displaying, via the computer device, the identified genotype at the target marker of the organism.

### CROSS-REFERENCE TO RELATED APPLICATION

This application claims the benefit of, and priority to, U.S. Provisional Application No. 63/540,038, filed on Sep. 22, 2023. The entire disclosure of the above-referenced application is incorporated herein by reference.

### FIELD

The present disclosure generally relates to systems and methods for use in resolving genotypic indicators, and in particular, to systems and methods for use in interpreting indicators associated with sample genomes, as indicative of target allele(s) in the sample genomes.

### BACKGROUND

This section provides background information related to the present disclosure which is not necessarily prior art.

Plants, animals, and other organisms are bred and raised for commercial and scientific purposes. Conventional breeding techniques for improving plant and animal stocks rely on controlled mating or crossing of parents, in which each parent conveys a given allele to produce at least one organism including the relevant alleles in a single genome. Among organisms with diploid or polyploid genomes, productions of a true-breeding stock with the requisite combination of alleles requires not merely that the alleles be found in the single genome, but that the allele for each locus in question be found on both or all chromosome sets (for diploid and polyploid organisms, respectively). This may require a substantial number of crosses, depending on the number of traits that need to be introgressed into a given stock, and verification of the specific alleles in the genome.

### SUMMARY

This section provides a general summary of the disclosure and is not a comprehensive disclosure of its full scope or all its features.

Example embodiments of the present disclosure generally relate to methods systems for use in resolving indicators associated with specific genotypes of samples. In one example embodiment, such a method generally includes exposing a sample of an organism to multiple probes, the multiple probes including a first type of probe configured to bind to a first target region containing a target marker with a first target allele and a second type of probe configured to bind to a second target region containing the target marker with a second target allele, the first allele different than the second allele; detecting, by a detector, intensities of fluorescence in the sample from the multiple probes; identifying, by a computer device, based on the detected intensities of fluorescence in the sample and using a generative model, a genotype at the target marker of the organism; and displaying, via the computer device, the identified genotype at the target marker of the organism.

Example embodiments of the present disclosure also generally relate to systems comprising computing devices configured to perform one or more of the above operations. Example embodiments of the present disclosure further relate to non-transitory computer-readable media including executable instructions, which, when executed by at least one processor, cause the at least one processor to perform one or more of the above operations.

Further areas of applicability will become apparent from the description provided herein. The description and specific examples in this summary are intended for purposes of illustration only and are not intended to limit the scope of the present disclosure.

### DRAWINGS

The drawings described herein are only for illustrative purposes of selected embodiments and are not intended to limit the scope of the present disclosure.

FIG. 1 is a block diagram of an example system of the present disclosure suitable for use in resolving indicators associated with target allele(s) in sample genomes;

FIGS. 2A-2C illustrate example graphical representations of the genotype categories that may be generated in the system of FIG. 1 and displayed to a user;

FIG. 3 is a block diagram of an example computing device that may be used in the system of FIG. 1; and

FIG. 4 is an example method, suitable for use with the system of FIG. 1, for resolving indicators associated with target allele(s) in a sample genome.

Corresponding reference numerals indicate corresponding parts throughout the several views of the drawings.

### DETAILED DESCRIPTION

Example embodiments will now be described more fully with reference to the accompanying drawings. The description and specific examples included herein are intended for purposes of illustration only and are not intended to limit the scope of the present disclosure.

Plants, animals and other organisms are bred and/or modified to fulfill commercial or scientific purposes. Genome editing techniques, for example, aim to accelerate the process of introducing traits into genomes. There are a number of techniques by which a genome may be edited. In connection therewith, it is problematic or difficult to verify the genome, after editing or otherwise, as being consistent with expectations, based on the details of the experiment, interpretation of the signals (e.g., from nucleotide fluorescence probes, etc.), or time and/or resources associated with verifying the genome.

Uniquely, the systems and methods herein provide for resolving indicators associated with specific target allele(s) in sample genomes, through use of multi-layer processes, based on a prediction of the target alleles in the sample genomes and estimation of uncertainty associated with the target alleles' prediction. In this manner, issues associated with the verification of the specific sample genome are reduced, whereby identification of the target alleles may be available in less time and accuracies of such identification may be improved.

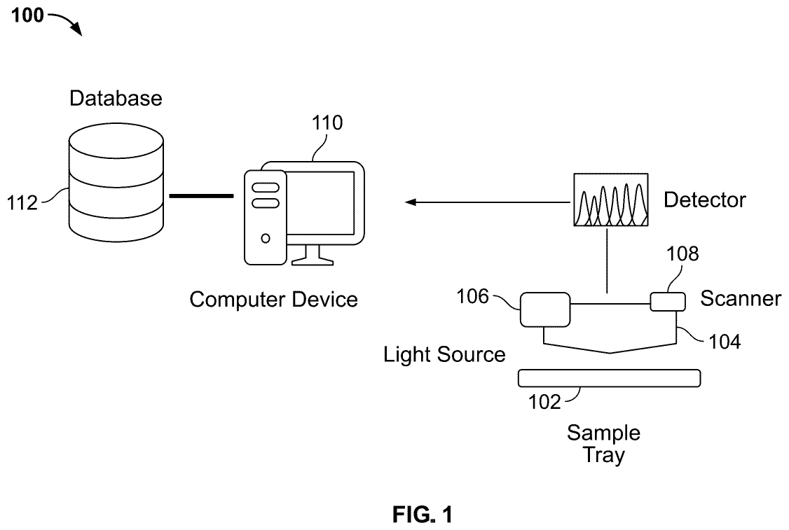

FIG. 1 illustrates an example system 100 in which the one or more aspects of the present disclosure may be implemented. Although the system 100 is presented in one arrangement, other embodiments may include the parts of the system 100 (or additional parts) arranged otherwise depending on, for example, the manner in which the multiple sample genomes are modified, selected, and/or tested to verify the expected genotype at one or more locus (i.e., marker) in the sample genomes.

In the example embodiment of FIG. 1, the system 100 generally includes a sample tray 102, which includes one or more sample genomes from one or more organisms, and also a scanner 104 disposed in association with the sample tray 102.

In particular, one or more organisms, or parts thereof, are identified for genotyping, based on, for example, a desired modification of the genome of the organism(s). The genotyping, then, may be employed to verify the specific genetic sequence or target allele at a target marker/locus that is present in the sample genome of the organism(s), for example, to verify a specific edit, inclusion of a specific sequence, exclusion of a specific sequence, etc. In connection therewith, the sample tray 102 (e.g., each well thereof, etc.) is populated with tissue samples or “sample genomes” from the organism(s), one per well of the sample tray 102, where the sample genomes may be from the same organism or different organisms. In this example embodiment, the organism(s) is (are) diploid(s), whereby each sample genome includes two copies of the DNA. In other embodiments, the organism(s) may be haploid or polyploid.

In this example embodiment, the sample genomes include a target allele at a target marker. A marker (i.e., locus) may be a nucleotide sequence with a known location (e.g., a chromosomal or genomic location). Markers may be used to identify differences within organisms, species, etc., and may include alternative nucleotide sequences called alleles. The alleles may include, for example, single nucleotide polymorphisms (SNPs), insertions, deletions, etc.

A target region of the DNA, that includes the target marker, of each of the sample genomes is amplified (e.g., by polymerase chain reaction (PCR), isothermal whole-genome amplification, etc.) in the system 100 to generate numerous copies of the target region of each of the sample genomes. In other embodiments, the sample genomes may include RNA or mRNA, whereby conversion (e.g., via RT-PCR into cDNA, etc.) may be employed prior to or after amplifying the target region. The target region copies are exposed, within the scanner 104, to various probes, for example, on a microarray beadchip, as explained below. The probes are specific to one or more nucleotide(s) (including a target allele at a target marker) in a target region(s) and designed to bind to that specific one or more nucleotide(s) (including the specific allele at the target marker) in the target region(s) (e.g., the probes define complimentary base pairs for all or some nucleotides in the target regions, etc.). Consequently, when the copies of the target region are exposed to the probes, the copies of the target region are hybridized to the probes (e.g., based on the complimentary pairs, etc.) if the target alleles at the target markers are present in the copies of the target region.

In particular, for the diploid organism, a set of probes, which includes two different types of probes (i.e., complementary to different base pairs, etc.), is directed to the target region, in which a target allele at a target marker (e.g., at an SNP, etc.) is expected to be located. The first type of probe is expected to bind to a target marker with target allele (X) (and emit one color of florescence), and the second type of probe is expected to bind to a target marker with target allele (Y) (and emit another color of florescence). In other words, each set of the probes genotype the target marker using two different color readouts: one color for each target allele (X and Y) at the target marker. The relative intensities of the two colors emitted is then indicative of whether the target marker, in the two copies of the DNA, is within a category of heterozygous (X,Y) or homozygous (X,X or Y,Y). It should be appreciated that the assay may be multiplexed to detect multiple target markers in each of multiple sample genomes.

With continued reference to FIG. 1, in this example embodiment, the scanner 104 is configured to illuminate the copies of the target region, exposed to the probes, with a light source 106 (e.g., a laser and wavelength filter, etc.) at a specific wavelength, whereby, based on the light and binding of the probes to the DNA in the target region, the fluorescent properties of the probes (e.g., fluorescence intensities, etc.) change (e.g., the probe emits a fluorescent glow, having a particular intensity, at a specific wavelength/color, etc.). In one specific embodiment, a Taqman assay may be used where the fluorogenic probes include a fluorescent reporter dye (e.g., FAM, VIC, etc.) attached to the 5′ end of the probe, and a quencher molecule attached to 3′ end of the probe. It should be appreciated that one or more additional binding agents may optionally be added to the quencher molecule. The probes are structured to anneal to the complementary target region, which may include a target allele, of the sample genome, and during PCR amplification, the Taq polypermase enzyme cleaves the probes, causing the reporter and quencher molecules to separate, which then causes the fluorescent emission (i.e., the fluorescent emission is prevented until the probe is cleaved by the Taq polymerase enzyme). For example, when the probe is intact, the proximity of the quencher molecule to the fluorescent reporter dye suppresses the reporter fluorescence. The cleaving results in an increase in fluorescence, which is in turn directly proportional to the amount of target region DNA copies that include the target allele. Thus, the fluorescence generated by amplification of the target region may indicate which target alleles are present at target markers in the sample genome.

In connection therewith, a detector 108 of the scanner 104 is positioned and configured to detect the fluorescence emitted from the probes, for example, on a beadchip (e.g., an intensity/color of the fluorescence from the different types of probes, etc.). The scanner 104 is then configured to compile output data indicative of the detected fluorescence from the probes over one or more amplification cycles (e.g., PCR amplification). The data output includes a listing of fluorescence intensities, separated by color, detected by the scanner 104. In connection therewith, the scanner 104 may record high-resolution images of the light emitted from the fluorophores. An example scanner suitable for use in the system 100 may include the Illumina iScan, BeadArray Reader, etc.

It should be appreciated that the probes may include control observations, whereby the target allele at a target marker is known and labeled. The detector 108 is configured to detect the intensities associated with the target allele control, and the target allele control intensities are included as part of the output data.

As shown in FIG. 1, the scanner 104 is configured to provide the output data to a computer device 110, which, in turn, is coupled in communication with a database 112. The computer device 110 is configured to store the output data in the database 112.

With continued reference to FIG. 1, in assessing the output data, the computer device 110 is configured to employ a generative model to determine the genotype category of each of the sample genomes at the target marker(s), as indicated by the intensities for each color, from the output data. The genotype categories may include homozygous_X (e.g., the target region (including the target allele “X”) binds to a first type of probe in both copies of the DNA, etc.), homozygous_Y (e.g., the target region (including the target allele “Y”) binds to a second type of probe in both copies of the DNA, etc.), or heterozygous (X,Y) (e.g., the target region (including the target allele “X”) binds to the first type of probe in one copy of the DNA and the target region (including the target allele “Y”) binds to the second type of probe in the other copy of the DNA), etc.

In connection therewith, the output data may form part of a training set, or after training, data to be categorized through use of the trained model.

As for training, in general, the output data is associated with annotations, either through a manual annotation processor or an automated annotation process. The training set then includes the intensities along with the annotation (as ground truths for the intensities). The annotations include, specifically, in this example, the genotype category of the sample genome at a target marker, from the example options above. The training set may include a portion, which is reserved from training, in order to validate the model after training.

With the training set defined, the computer device 110 is configured to then train a generative model, based on the training set, to define a genotype category for intensities. In particular, in this embodiment, the computer device 110 is configured to train the example generative model represented in Equation 1, below.

$${Y}_{i j} \vert {π}_{i j k} ∼ Multinom ( {π}_{i j 1} , {π}_{i j 2} , {π}_{i j 3} ) where {π}_{i j k} := \frac{{ϵ}^{g ( {X}_{i j} {β}_{k} )}}{{\sum }_{a = 1}^{3} {ϵ}^{g ( {X}_{i j} {β}_{a} )}} \qquad ( 1 )$$

In Equation 1, Xᵢⱼ∈R²$ is the predictor for the j-th sample genome (i.e., sample) at the i-th target marker (i.e., marker), g( ) represents some given function (as described more below), and πᵢⱼₖ is the probability of the j-th sample at the i-th marker being from the class k∈{het, hom_X, hom_Y}. In training the generative model, based on the training set, specifically, the computer device 110 is configured to determine the values of βₐ, βₖ.

Further, in this example embodiment, the probes included in the scanner 104 may include control observations, which may be used to tune the model. The control observations are based on a population of samples, which are interrogated by the probes (as described herein) and also known to included specific genomes. The control observations at each marker are labeled samples, which are used herein as part of the re-centering parameter γⱼ˜R^{2×2} which scales and rotates the control observations to match the general generative model provided above. Specifically, in this example embodiment, where the observations are re-centered, the values of Bu, BR are unchanged or retained from the training described above despite the control observations. The two layered approach proceeds to recenter, scale and/or rotate, broadly, reshape, the observation inputs (e.g., X, etc.) through Equations 2 and 3, below.

$${Y}_{i j} \vert γ j , {π}_{i j k} ∼ Multinom ( {π}_{i j 1} , {π}_{i j 2} , {π}_{i j 3} ) where {π}_{i j k} := \frac{{ϵ}^{g ( {X}_{i j} γ j {β}_{k} )}}{{\sum }_{a = 1}^{3} {ϵ}^{g ( {X}_{i j} γ j {β}_{a} )}} \qquad ( 2 )$$

$${γ}_{i} \vert X ∼ {Multinomal}_{2 \times 2} ( {\hat{γ}}_{i} , {σ}^{2} I ) \qquad ( 3 )$$

$${\hat{γ}}_{i} = {( {X}_{c}^{( j ) T} {X}_{c}^{( j )} )}^{- 1} {X}_{c}^{( j ) T} \overline{X}$$

In Equations 2 and 3, X_{c}^{(j)T}∈R^{|cj|×2} is the matrix of control intensities, |cj| is the number of controls at marker j, and X̅∈R^{|cj|×2} represents the expected center of mass of the intensities for each category, respective to each control. In this way, based on the control observations, the trained model in this example may be translated, in order to reduce the inference overhead, to a simple dot product between the observations and the re-centering parameter y, as a manner of reshaping the inputs of the equation (e.g., X, etc.). In an alternative example, the computer device 110 may be configured to use a non-linear mapping between the control observations and the recentering parameter γ, as a manner of reshaping the inputs of the equation.

As part of the above, it may further be assumed, in one or more embodiments, that intensities of the control observations may not be reliable.

In connection therewith, for each marker, X∈R^{|cj|×2} is the matrix of observed intensities of the control, and Ŷ∈{0,1}^{n×2} is the design matrix (e.g., one-hot encoded or other techniques for numerical representation of categories, etc.) of the predicted categories (HOM_{X}, HOM_{Y}, HET). The matrix of the predicted intensities of the controls in then defined as \hat{X}:=X[X^{T} X]⁻¹ XY. Also, the difference between observed and predicted, is then defined as E:=X−\hat{X}∈R^{n×p}. Given that, the goodness of the fit of a given scoring run is defined in Equation (5), below:

$$\begin{matrix}{r}^{2} = {p}^{- 1} T r [ 1 - diag ( {E}^{T} E ) ( diag {( {X}^{T} X )}^{- 1} ] \\ {r}_{adj}^{2} = 1 - ( 1 - {r}^{2} ) [ \frac{n - 1}{n - p - 1} ]\end{matrix} \qquad ( 5 )$$

where r_{adj}²∈[0,1] is R-squared adjusted, 1∈R^{n×2}_ is the matrix of ones, and n is the number of samples.

Based on the above, the computer device 110 is configured to impose a control skepticism procedures, whereby the scoring interference to determined, consistent with Equations (1)-(4) above, and then to compute r_{adj}² values, and if r_{adj}² is below a certain threshold, to determine the observed intensities of the controls to be unreliable.

Next, the computer device 110 is configured to cluster the data, via one or more clustering techniques, such as, for example, the k-means clustering technique for vector quantification, etc. In this example, the clustering is based on Equation (6), below.

$$Z , M = KMeans ( X ) \qquad ( 6 )$$

where Z∈R^{k×k} is the matrix of centroids and M∈R^{n×k} is the matrix of cluster membership, and k being the number of clusters. The number of clusters k is determined through an elbow method, which include plotting explained variation as a function of number of clusters and then selecting the “elbow” of the curve as the number of clusters k to be used. The computer device 110 is configured to then run the scoring on the centroids using Equation (1), and further ignoring the controls, based on Equation (7):

$${\hat{Y}}_{Z} = Autoscoring ( Z ) \qquad ( 7 )$$

where Ýz∈[0,1]^{k×2} is the predicted categories of the centroids. The categories are again, defined above, i.e., HOM_{X}, HOM_{Y}, and HET. The computing device 110 is configured to propagate to the samples, through Equation (8).

$$\hat{Y} = M {\hat{Y}}_{Z} \qquad ( 8 )$$

where Ŷ_{Z}∈[0,1]^{n×2} is the predicted categories for the samples. Based on the above, the computing device 110 is configured to then employ the reliable control observations in the training set, as explained above, to train the model, while the unreliable control observations are discarded. In this way, the computing device 110 is configured to efficiently control situations in which the control intensities are not reliable.

In connection with the above, the model is generic to multiple different functions, g( ). As such, the computer device 110 may be configured to employ different functions g( ) in training the model. Specifically, in this example embodiment, the function g( ) may include, for example, a parametric or non-parametric function. For a parametric function, the function g( ) may include a linear multinomial having one of the following settings: additive (HOM_{X}+HOM_{Y}), full interaction (HOM_{X}+HOM_{Y}+HOM_{X}×HOM_{Y}), singular value re-scaling (γ(HOM_{X}+HOM_{Y}), where γ is the largest singular value of a given marker plate covariance matrix), or linear normalized (HOM\dot{x}+HOM\dot{y}). For a non-parametric function, the function g( ) may include a decision tree or a version of a multilayer perceptron (MLP) (e.g., having two layers, three layers or five layers, etc.).

It should be appreciated that other functions g( ) may be included in the models above in other embodiments.

Once the generative model is trained, the computer device 110 is configured to use the reserved portion of the training set to validate the trained model. When the validation of the trained model indicates a sufficient performance, the computer device 110 is configured to store the generative model in the database 112.

Thereafter, additional sample genomes are included in the sample tray 102 (i.e., to confirm target alleles being present therein) (consistent with the above), and target regions of the sample genomes are amplified and exposed to the above probes in the scanner 104, whereby the detector 108, consistent with the above, is configured to detect the fluorescence emitted from the probes (e.g., an intensity/color of the fluorescence from the different types of probes, etc.). The scanner 104 is configured to compile output data indicative of the detected fluorescence intensities from the probes. The scanner 104 is configured to provide the output data to the computer device 110, which in turn, is configured, by the generative model, to predict the genotype category of each of the target markers in each of the sample genomes as one of: homozygous_X for a first allele, homozygous_Y for a second allele, or heterozygous between the two alleles.

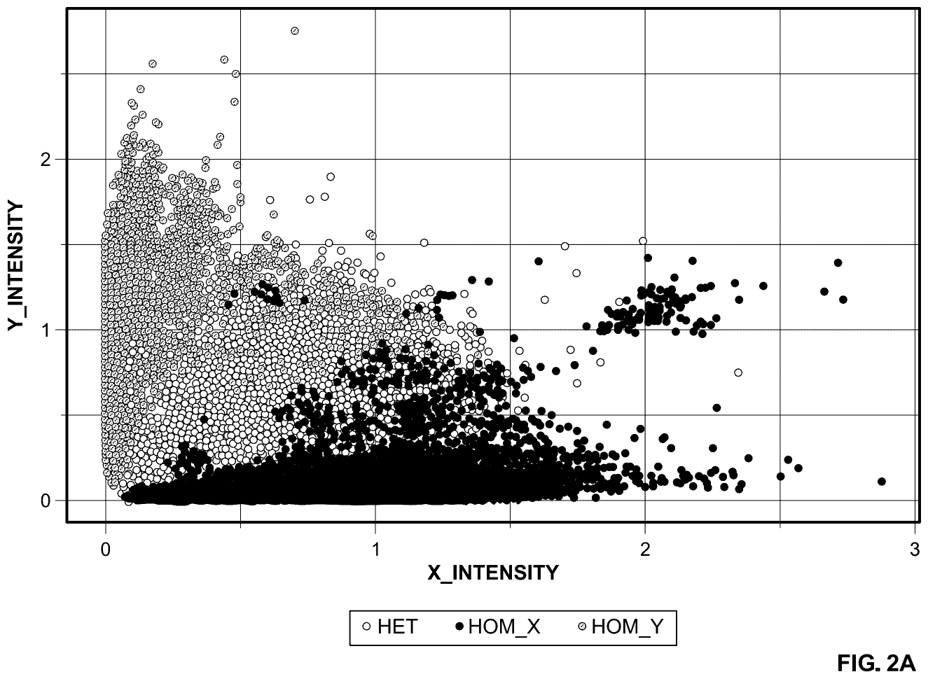

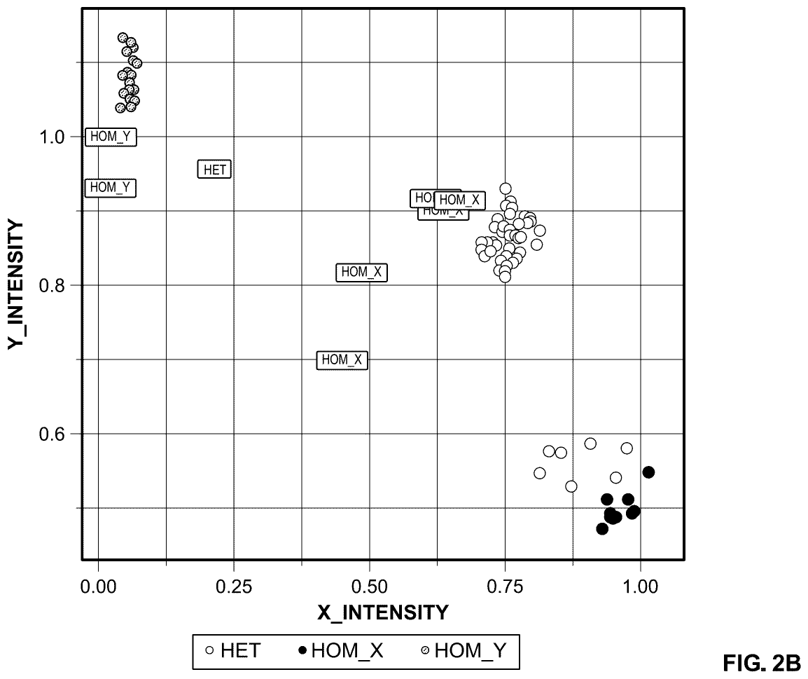

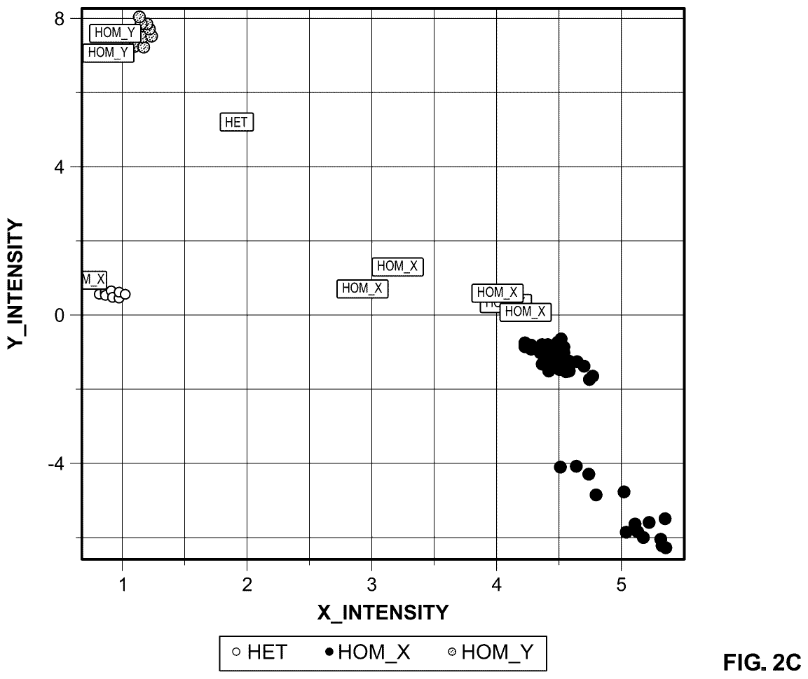

Further, the computing device 110 is configured to display a graphical representation of the genotype categories of the additional target markers in the additional sample genomes. FIG. 2A, for example, displays target allele data for multiple different target markers from multiple sample genomes. FIG. 2A illustrates a graphical representation of the intensities of the fluorescence of the additional target markers, and also the categories of the target markers, as indicated by the circles with different hatch/fill patterns, where HOM_{Y} are represented by a dotted fill pattern, HOM_{X} are represented by a solid fill pattern, and HET are represented by no fill pattern. FIG. 2A indicates high variability in the fluorescence intensity for all target alleles across both target markers and sample genomes. It should be appreciated that one or more other graphical representations of the categories and/or intensities may be included in other embodiments.

It should be appreciated that considering the control observation, as described above, to reshape the input observation may alter the specific genotype category and/or the overall accuracy of the generative model. FIGS. 2B and 2C show multiple sample genomes at a target marker before and after recentering relative to the sample controls. As shown between FIGS. 2B and 2C, the reshaping of the input observation substantially impacts the genotype categories of the additional sample genomes. Again, in FIGS. 2B and 2C, the genotype categories of the sample genomes at the target markers are indicated by the circles having different hatch/fill patterns, for example, where HOM_{Y} are represented by a dotted fill pattern; HOM_{X} are represented by a solid fill pattern; and HET are represented by no fill pattern.

Based on the predicted genotype category, the system 100 verifies the target allele at the target marker in the given sample genome, and designates the organism to be advanced, or not, consistent with the predicted presence or absence of the target allele based on the genotype category. The system 100 may use a Fingerprinting (FP) assay, for instance, for scoring about 40-60 thousand markers per sample. In another embodiment, the system 100 may use Marker Assisted Selection (MAS). In any case, the system 100 permits a user to advance the organisms as being consistent with an expected genotype category, or not, based thereon. That said, genotype categories may be used for advancement as described (e.g., via the MAS workflow, etc.), or to characterize the sample genome to advance progeny of these characterized lines (e.g., via the FP assay workflow, etc.).

FIG. 2 illustrates an example computing device 200 that can be used in the system 100. The computing device 200 may include, for example, one or more servers, workstations, computers, laptops, tablets, smartphones, etc. In addition, the computing device 200 may include a single computing device, or it may include multiple computing devices located in close proximity or distributed over a geographic region, so long as the computing devices are specifically configured to operate as described herein.

In the example embodiment of FIG. 1, each of the sample tray 102 and the scanner 104 may include one or more computing devices consistent with computing device 200. Also, in the example embodiment, the system 100 includes the computer device 110 and the database 112, each of which may be understood to be consistent with the computing device 200 and/or implemented in a computing device consistent with computing device 200 (or a part thereof, such as, for example, memory 204, etc.). However, the system 100 should not be considered to be limited to the computing device 200, as described below, as different computing devices and/or arrangements of computing devices may be used. In addition, different components and/or arrangements of components may be used in other computing devices.

As shown in FIG. 2, the example computing device 200 includes a processor 202 and a memory 204 coupled to (and in communication with) the processor 202. The processor 202 may include one or more processing units (e.g., in a multi-core configuration, etc.). For example, the processor 202 may include, without limitation, a central processing unit (CPU), a microcontroller, a reduced instruction set computer (RISC) processor, a graphics processing unit (GPU), an application specific integrated circuit (ASIC), a programmable logic device (PLD), a gate array, and/or any other circuit or processor capable of the functions described herein.

The memory 204, as described herein, is one or more devices that permit data, instructions, etc., to be stored therein and retrieved therefrom. In connection therewith, the memory 204 may include one or more computer-readable storage media, such as, without limitation, dynamic random access memory (DRAM), static random access memory (SRAM), read only memory (ROM), erasable programmable read only memory (EPROM), solid state devices, flash drives, CD-ROMs, thumb drives, floppy disks, tapes, hard disks, and/or any other type of volatile or nonvolatile physical or tangible computer-readable media for storing such data, instructions, etc. In particular herein, the memory 204 is configured to store data including, without limitation, genome sequences, models, and/or other types of data (and/or data structures) suitable for use as described herein. Furthermore, in various embodiments, computer-executable instructions may be stored in the memory 204 for execution by the processor 202 to cause the processor 202 to perform one or more of the operations described herein in connection with the various different parts of the system 100 (e.g., one or more of the operations of method 300, etc.), such that the memory 204 is a physical, tangible, and non-transitory computer readable storage media. Such instructions often improve the efficiencies and/or performance of the processor 202 that is performing one or more of the various operations herein and, in connection with such performance, transform the computing device 200 into a special purpose computing device. It should be appreciated that the memory 204 may include a variety of different memories, each implemented in connection with one or more of the functions or processes described herein.

In the example embodiment, the computing device 200 also includes a presentation unit 206 that is coupled to (and that is in communication with) the processor 202 (however, it should be appreciated that the computing device 200 could include output devices other than the presentation unit 206, etc.). The presentation unit 206 may output information (e.g., predictions as to the genome, etc.), visually to a user of the computing device 200, such as a breeder or other person associated with selection of a nature of edits, etc. It should be further appreciated that various interfaces (e.g., as defined by network-based applications, websites, etc.) may be displayed at computing device 200, and in particular at presentation unit 206, to display certain information to the user. The presentation unit 206 may include, without limitation, a liquid crystal display (LCD), a light-emitting diode (LED) display, an organic LED (OLED) display, an “electronic ink” display, speakers, etc. In some embodiments, presentation unit 206 may include multiple devices. Additionally or alternatively, the presentation unit 206 may include printing capabilities, enabling the computing device 200 to print text, images, and the like on paper and/or other similar media.

In addition, the computing device 200 includes an input device 208 that receives inputs from the user (i.e., user inputs) such as, for example, selections of organisms to genotype, etc. The input device 208 may include a single input device or multiple input devices. The input device 208 is coupled to (and is in communication with) the processor 202 and may include, for example, one or more of a keyboard, a pointing device, a mouse, a stylus, a touch sensitive panel (e.g., a touch pad or a touch screen, etc.), or other suitable user input devices. In addition, the input device 208 may include, without limitation, settings associated with the scanner 104, such as, for example, defining target regions, etc. It should be appreciated that in at least one embodiment an input device 208 may be integrated and/or included with presentation unit 206 (e.g., a touchscreen display, etc.).

Further, the illustrated computing device 200 also includes a network interface 210 coupled to (and in communication with) the processor 202 and the memory 204. The network interface 210 may include, without limitation, a wired network adapter, a wireless network adapter, a mobile network adapter, or other device capable of communicating to one or more different networks (e.g., one or more of a local area network (LAN), a wide area network (WAN) (e.g., the Internet, etc.), a mobile network, a virtual network, and/or another suitable public and/or private network capable of supporting wired and/or wireless communication among two or more of the parts illustrated in FIG. 1, etc.), including with other computing devices used as described herein.

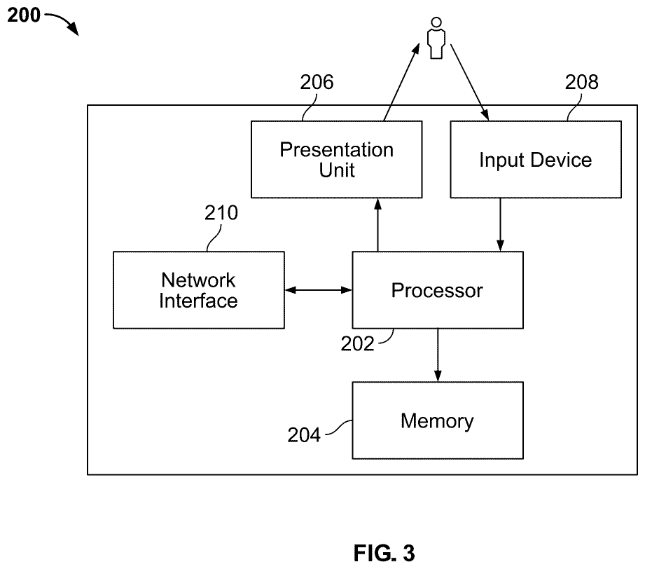

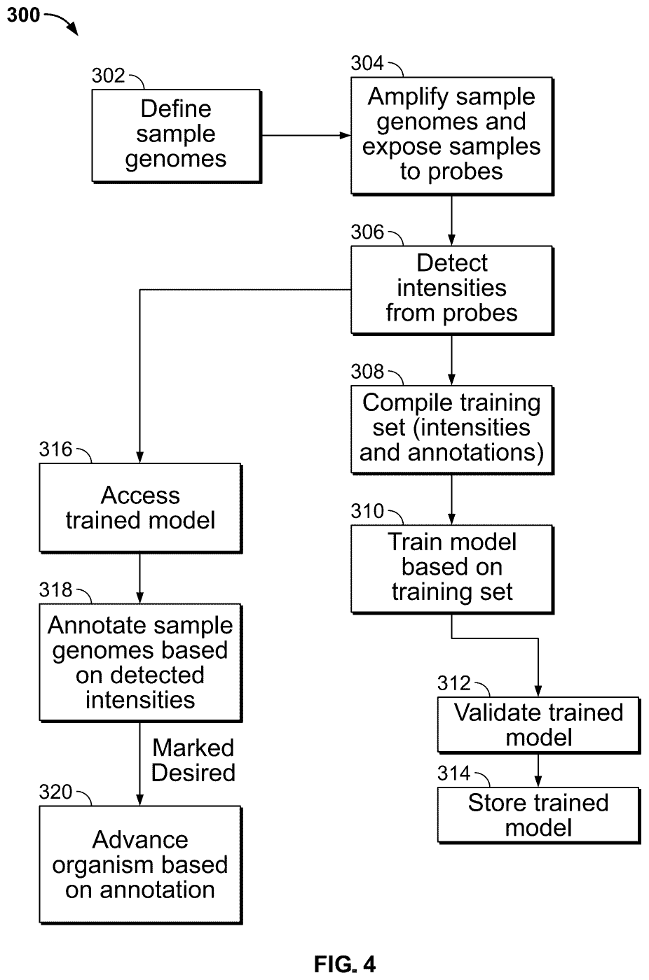

FIG. 3 illustrates an example method 300 for use in resolving indicators associated with specific genotypes of samples. The example method 300 is described herein in connection with the computer device 110 of the system 100, and is also described with reference to computing device 200. However, it should be appreciated that the methods herein are not limited to the system 100, or computing device 200. And, likewise, the systems and computing devices described herein are not limited to the example method 300.

At the outset, various organisms are associated with the method 300, whereby a number of organisms include known genotypes or alleles and others include unknown genotypes or alleles, for example, based on modification of the genomes. In general, there is an allele of interest (i.e., target allele) at a target marker in the DNA of an organism. The presence or absence of the allele in one or more copies of the DNA defines a genotype category for the organism. As such, the method 300 aims at resolving the indicators of the target allele (e.g., as defined by the intensities from probes exposed to the sample genome, etc.) to predict the particular genotype category of the organism.

Initially, however, in the method 300, a number of sample genomes are defined, at 302, including, potentially, sample genomes (which may be part of or may not be part of an organism, or may be part of the same type of organism (e.g., corn plants, etc.) or different organisms, etc.) having a known target allele or an unknown target allele at a specific target marker. The sample genomes are physically deposited in the sample tray 102, for example, and introduced to the scanner 104.

In connection therewith, at 304, target regions (including the target marker) of the sample genomes are amplified and exposed to probes included in the scanner 104, as described above. The target regions are amplified to generate numerous copies of the DNA. In addition to amplifying the target regions, the sample genomes are also denatured in this embodiment, to pull apart the DNA strands of the diploid organism. As further explained above, the probes of the scanner 104 are designed to bind to a specific portion of the target region, that includes the target marker/locus and the target allele. In addition, the probes of the scanner 104 are designed to emit a certain color or fluorescence, when bound to target region including the target allele of the DNA, and illuminate with the light source 106.

As such, upon exposure of the samples to the probes, and application of the light (e.g., from the light source 106, etc.), the probes emit the fluorescence, at a particular intensity. The detector 108 of the scanner 104 detects, at 306, the intensities of the fluorescence, for specific colors, emitted from the probes. The intensities of the fluorescence for each of the colors are represented as output data from the detector 108.

It should be appreciated that the intensities of the fluorescence are inspected and/or associated with the known target alleles of the organism, at the target marker/locus, to annotate the sample genomes with a specific genotype category as being one of: homozygous as to a specific allele, homozygous as to a different allele, or heterozygous. The intensities of the fluorescence and the annotations for the different sample genomes are compiled, at 308, into a training set. A portion of the training set is then reserved as a validation set.

At 310, the computer device 110 trains the generative model, based on the training set, which may be based on Equation 1 where control observations are not to be considered (e.g., control observations are unavailable, etc.), or based on Equations 1-3, where control observations are to be used. It should be appreciated that the computer device 110 may further rely on Equations 5-8 to confirm the reliability of the control intensities in the training set in connection with the above.

It should be appreciated, also, that the Equations 1-3 generally rely on a function g( ) and that one or more of the different functions noted above may be included as part of the generative model. After training, the computer device 110 validates, at 312, the trained model for each of the one or more different functions g( ) to identify the performance of the trained generative model for a specific function g( ) or for multiple models each specific to one of the functions g( ). The validation may be based on a part of the training set, which was reserved, and not used in training. One of the models, for example, a model with the highest validation rate (e.g., a percentage of the marker predicted correctly, based on the respective intensities, in the validation portion of the training set, etc.), is then stored, at 314, in the database 112 by the computer device 110.

Thereafter, for a target organism for which the specific inclusion or not of a target allele, for example, is unknown, the method 300 includes defining a sample genome, at 302 for that target organism. Consistent with the above, at 304, a part of the sample genome (i.e., the target region) is amplified and exposed to probes in the scanner 104, as described above. The target region is amplified to generate numerous copies of the DNA, and also denatured in this embodiment, to pull apart the DNA strands of the diploid organisms. Upon exposure of the samples to the probes, and application of the light (e.g., from the light source 106, etc.), the probes emit the fluorescence, at a particular intensity. The detector 108 of the scanner 104 detects, at 306, the intensities from the probes. The intensities are represented as output data from the detector 108.

As shown in FIG. 3, in this implementation, at 316, the computer device 110 accesses the stored trained generative model and, at 318, annotates a genotype category to a target marker in the sample genome, based on the detected intensities of the fluorescence, using the trained generative model.

Thereafter, with the annotation, as either homozygous as to a specific allele, homozygous as to a different allele, or heterozygous, the organism is advanced, at 320, when the annotation of the specific category is desired. Alternatively, although not shown, the organism may be discarded when the annotation is not as desired.

In view of the above, the systems and methods herein provide for rapid and accurate resolution of indicators of specific target markers in an organism, whereby the sample genome of the organism is categorized as an indication of specific target alleles being present, or not (i.e., having a certain genotype at a target marker). Consistent with the systems and methods described herein, the categorization of sample genomes may be substantially more rapid (e.g., may have about 200×-300× faster performance than a legacy technique (e.g., using support vector machines, etc.), etc.), while demonstrating a greater than about 99% accuracy (e.g., as verified through manual review, etc.) (as compared to about 97% accuracy by the legacy technique), etc. The systems and methods herein also provide for substantial decreases in runtime for experiments, while providing enhanced accuracy. This may be of value to genotyping laboratories, for example, to decrease analytic turnaround time (e.g., from multiple hours to a few minutes, etc.) on various high marker density genotyping projects. High marker density assays, for example, may include a Fingerprinting assay with about 10 thousand or more markers, depending on the organism type.

The functions described herein, in some embodiments, may be described in computer executable instructions stored on a computer readable media, and executable by one or more processors. The computer readable media is a non-transitory computer readable media. By way of example, and not limitation, such computer readable media can include RAM, ROM, EEPROM, CD-ROM or other optical disk storage, magnetic disk storage or other magnetic storage device, or any other medium that can be used to carry or store desired program code in the form of instructions or data structures and that can be accessed by a computer. Combinations of the above should also be included within the scope of computer-readable media.

It should also be appreciated that one or more aspects of the present disclosure transform a general-purpose computing device into a special-purpose computing device when configured to perform the functions, methods, and/or processes described herein.

As will be appreciated based on the foregoing specification, the above-described embodiments of the disclosure may be implemented using computer programming or engineering techniques including computer software, firmware, hardware or any combination or subset thereof, wherein the technical effect may be achieved by performing at least one of the following operations: (a) exposing a sample of an organism to multiple probes, the multiple probes including a first type of probe configured to bind to a first target region containing a target marker with a first target allele and a second type of probe configured to bind to a second target region containing the target marker with a second target allele, the first allele different than the second allele; (b) detecting, by a detector, intensities of fluorescence in the sample from the multiple probes; (c) identifying, based on the detected intensities of fluorescence in the sample and using a generative model, the genotype at the target marker of the organism; and/or (d) displaying the identified genotype at the target marker of the organism.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. In addition, advantages and improvements that may be achieved with one or more example embodiments disclosed herein may provide all or none of the above mentioned advantages and improvements and still fall within the scope of the present disclosure.

Specific values disclosed herein are example in nature and do not limit the scope of the present disclosure. The disclosure herein of particular values and particular ranges of values for given parameters are not exclusive of other values and ranges of values that may be useful in one or more of the examples disclosed herein. Moreover, it is envisioned that any two particular values for a specific parameter stated herein may define the endpoints of a range of values that may also be suitable for the given parameter (i.e., the disclosure of a first value and a second value for a given parameter can be interpreted as disclosing that any value between the first and second values could also be employed for the given parameter). For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises”, “comprising”, “including”, and “having” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When a feature is referred to as being “on”, “engaged to”, “connected to”, “coupled to”, “associated with”, “in communication with”, or “included with” another element or layer, it may be directly on, engaged, connected or coupled to, or associated or in communication or included with the other feature, or intervening features may be present. As used herein, the term “and/or” and the phrase “at least one of” includes any and all combinations of one or more of the associated listed items.

Although the terms first, second, third, etc. may be used herein to describe various features, these features should not be limited by these terms. These terms may be only used to distinguish one feature from another. Terms such as “first”, “second”, and other numerical terms when used herein do not imply a sequence or order unless clearly indicated by the context. Thus, a first feature discussed herein could be termed a second feature without departing from the teachings of the example embodiments.

None of the elements recited in the claims are intended to be a means-plus-function element within the meaning of 35 U.S.C. § 112 (f) unless an element is expressly recited using the phrase “means for,” or in the case of a method claim using the phrases “operation for” or “step for.”

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method for use in resolving indicators associated with specific genotypes of samples, the method comprising:
exposing a sample of an organism to multiple probes, the multiple probes including a first type of probe configured to bind to a first target region containing a target marker with a first target allele and a second type of probe configured to bind to a second target region containing the target marker with a second target allele, the first allele different than the second allele;
detecting, by a detector, intensities of fluorescence in the sample from the multiple probes;
identifying, by a computer device, based on the detected intensities of fluorescence in the sample and using a generative model, a genotype at the target marker of the organism; and
displaying, via the computer device, the identified genotype at the target marker of the organism.

2. The method of claim 1, wherein the organism includes a diploid organism; and
wherein the identified genotype at the target marker is one of: homozygous as to the first allele, homozygous as to the second allele, or heterozygous as to the first and second alleles.

3. The method of claim 1, wherein the multiple probes are included on a microarray beadchip.

4. The method of claim 1, further comprising:
training the generative model based on a first part of a training set; and
validating the trained generative model based on a second part of the training set, the first part of the training set being different than the second part of the training set; and
wherein identifying the genotype at the target marker of the organism includes identifying the allele(s) indicative of the genotype at the target marker of the organism using the trained generative model.

5. The method of claim 4, wherein the generative model is based on one or more of the following expressions:
$${Y}_{i j} \vert {π}_{i j k} ∼ Multinom ( {π}_{i j 1} , {π}_{i j 2} , {π}_{i j 3} ) where {π}_{i j k} := \frac{{ϵ}^{g ( {X}_{i j} {β}_{k} )}}{{\sum }_{a = 1}^{3} {ϵ}^{g ( {X}_{i j} {β}_{a} )}} .$$

6. The method of claim 5, further comprising re-shaping input to the generative model based on multiple control observations and the following expressions:
$${Y}_{i j} \vert γ j , {π}_{i j k} ∼ Multinom ( {π}_{i j 1} , {π}_{i j 2} , {π}_{i j 3} ) where {π}_{i j k} := \frac{{ϵ}^{g ( {X}_{i j} γ j {β}_{k} )}}{{\sum }_{a = 1}^{3} {ϵ}^{g ( {X}_{i j} γ j {β}_{a} )}}$$
$${γ}_{i} \vert X ∼ {Multinomal}_{2 \times 2} ( {\hat{γ}}_{i} , {σ}^{2} I )$$
$${\hat{γ}}_{i} = {( {X}_{c}^{( j ) T} {X}_{c}^{( j )} )}^{- 1} {X}_{c}^{( j ) T} \overline{X}$$

7. The method of claim 5, wherein the function g( ) includes one of a parametric function or a non-parametric function.

8. The method of claim 6, wherein the function g( ) includes one of: an additive linear multinomial, a full interaction linear multinomial, a singular value re-scaling linear multinomial, a linear normalized multinomial, or a multilayer perceptron (MLP) having two layers, three layers or five layers.

9. The method of claim 5, wherein at least part of the training set is assessed for reliability based on a K-means clustering of the multiple control observations.

10. A non-transitory computer-readable medium including executable instructions, which, when executed by at least one processor, cause the at least one processor to:
expose a sample of an organism to multiple probes, the multiple probes including a first type of probe configured to bind to a first target region containing a target marker with a first target allele and a second type of probe configured to bind to a second target region containing the target marker with a second target allele, the first allele different than the second allele;
detect, via a detector, intensities of fluorescence in the sample from the multiple probes;
identify, based on the detected intensities of fluorescence in the sample and using a generative model, a genotype at the target marker of the organism; and
display the identified genotype at the target marker of the organism.

11. The non-transitory computer-readable medium of claim 10, wherein the organism includes a diploid organism; and
wherein the identified genotype at the target marker is one of: homozygous as to the first allele, homozygous as to the second allele, or heterozygous as to the first and second alleles.

12. The non-transitory computer-readable medium of claim 10, wherein the multiple probes are included on a microarray beadchip.

13. The non-transitory computer-readable medium of claim 10, wherein the executable instructions, when executed by the at least one processor, further cause the at least one processor to:
train the generative model based on a first part of a training set; and
validate the trained generative model based on a second part of the training set, the first part of the training set being different than the second part of the training set; and
wherein the executable instructions, when executed by the at least one processor to identify the genotype at the target marker of the organism, cause the at least one processor to identify the allele(s) indicative of the genotype at the target marker of the organism using the trained generative model.

14. The non-transitory computer-readable medium of claim 10, wherein the generative model is based on one or more of the following expressions:
$${Y}_{i j} \vert {π}_{i j k} ∼ Multinom ( {π}_{i j 1} , {π}_{i j 2} , {π}_{i j 3} ) where {π}_{i j k} := \frac{{ϵ}^{g ( {X}_{i j} {β}_{k} )}}{{\sum }_{a = 1}^{3} {ϵ}^{g ( {X}_{i j} {β}_{a} )}} .$$

15. The non-transitory computer-readable medium of claim 14, wherein the executable instructions, when executed by the at least one processor, further cause the at least one processor to re-shape input to the generative model based on multiple control observations and the following expressions:
$${Y}_{i j} \vert γ j , {π}_{i j k} ∼ Multinom ( {π}_{i j 1} , {π}_{i j 2} , {π}_{i j 3} ) where {π}_{i j k} := \frac{{ϵ}^{g ( {X}_{i j} γ j {β}_{k} )}}{{\sum }_{a = 1}^{3} {ϵ}^{g ( {X}_{i j} γ j {β}_{a} )}}$$
$${γ}_{i} \vert X ∼ {Multinomal}_{2 \times 2} ( {\hat{γ}}_{i} , {σ}^{2} I )$$
$${\hat{γ}}_{i} = {( {X}_{c}^{( j ) T} {X}_{c}^{( j )} )}^{- 1} {X}_{c}^{( j ) T} \overline{X} .$$

16. The non-transitory computer-readable medium of claim 15, wherein the function g( ) includes one of a parametric function or a non-parametric function.

17. The non-transitory computer-readable medium of claim 15, wherein the function g( ) includes one of: an additive linear multinomial, a full interaction linear multinomial, a singular value re-scaling linear multinomial, a linear normalized multinomial, or a multilayer perceptron (MLP) having two layers, three layers or five layers.

18. A system for use in resolving indicators associated with specific genotypes of samples, the system comprising at least one computing device configured to:
expose a sample of an organism to multiple probes, the multiple probes including a first type of probe configured to bind to a first target region containing a target marker with a first target allele and a second type of probe configured to bind to a second target region containing the target marker with a second target allele, the first allele different than the second allele;
detect, via a detector, intensities of fluorescence in the sample from the multiple probes;
identify, based on the detected intensities of fluorescence in the sample and using a generative model, a genotype at the target marker of the organism; and
display the identified genotype at the target marker of the organism.

19. The system of claim 18, wherein the generative model is based on one or more of the following expressions:
$${Y}_{i j} \vert {π}_{i j k} ∼ Multinom ( {π}_{i j 1} , {π}_{i j 2} , {π}_{i j 3} ) where {π}_{i j k} := \frac{{ϵ}^{g ( {X}_{i j} {β}_{k} )}}{{\sum }_{a = 1}^{3} {ϵ}^{g ( {X}_{i j} {β}_{a} )}} .$$

20. The system of claim 19, wherein the at least one computing device is further configured to re-shape input to the generative model based on multiple control observations and the following expressions:
$${Y}_{i j} \vert γ j , {π}_{i j k} ∼ Multinom ( {π}_{i j 1} , {π}_{i j 2} , {π}_{i j 3} ) where {π}_{i j k} := \frac{{ϵ}^{g ( {X}_{i j} γ j {β}_{k} )}}{{\sum }_{a = 1}^{3} {ϵ}^{g ( {X}_{i j} γ j {β}_{a} )}}$$
$${γ}_{i} \vert X ∼ {Multinomal}_{2 \times 2} ( {\hat{γ}}_{i} , {σ}^{2} I )$$
$${\hat{γ}}_{i} = {( {X}_{c}^{( j ) T} {X}_{c}^{( j )} )}^{- 1} {X}_{c}^{( j ) T} \overline{X} .$$
